(19) United States
(12) Patent Application Publication
(10) Pub. No.: US 2009/0148464 A1
(43) Pub. Date: Jun. 11, 2009

(54) PREVENTIVE OR THERAPEUTIC AGENT CONTAINING ANTI THIOREDOXIN ANTIBODY POLYPEPTIDE AS AN ACTIVE INGREDIENT FOR DISORDERS

(76) Inventors: Junji YODOI, Kyoto-shi (JP); Aoi Son, Kyoto-Shi (JP); Norihiko Kondo, Yokohama-shi (JP); Hajime Nakamura, Osaka (JP); Tomijiro Hara, Higashimurayama-Gun (JP); Noriko Kato, Ashiya-shi (JP)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/033,295

(22) Filed: Feb. 19, 2008

(30) Foreign Application Priority Data
Dec. 11, 2007 (JP) 2007-320233

(51) Int. Cl. A61K 39/395; A61P 9/00

(52) U.S. Cl. 424/172.1

(57) ABSTRACT

The present invention provides a preventive or therapeutic agent being effective for disorders induced by macrophage migration inhibitory factor and vascular system disorders, and having fewer side effects. In particular, the present agent is characterized by containing anti TRX antibody polypeptide as an active ingredient to inhibit adherence of leukocyte to vascular endothelial cell and a cell internalization of the macrophage migration inhibitory factor.

This application claims benefit of Japanese Application No. JP 2007-320233 filed Dec. 11, 2007 under the Paris Convention. The entire content and disclosure of the preceding application is incorporated by reference into this application.

### FIELD OF THE INVENTION

The present invention relates to a preventive or therapeutic agent containing anti thioredoxin antibody polypeptide as an active ingredient, and more particularly, to a preventive or therapeutic agent for disorders induced by macrophage migration inhibitory factor and vascular system disorders.

### DESCRIPTION OF THE BACKGROUND ART

Thioredoxin (TRX) is a 12-kDa multifunctional polypeptide with an oxidizing-reducing (redox) activity via a disulfide/dithiol exchange reaction between 2 cysteine residues in the active site: [-Cys-Gly-Pro-Cys-] (Redox regulation of cellular activation Ann. Rev. Immunol. 1997;15:351-369). TRX has been isolated from and identified in many prokaryotes and eukaryotes since it was isolated from Escherichia coli as a coenzyme donating hydrogen ions to anribonucleotide reductase which is an enzyme essential for E. coli deoxyribonucleic acid (DNA) synthesis.

Adult T-cell leukemia-derived factor (ADF) was initially identified as an IL-2 receptor-inducing factor produced by HTLV-1-infected T-lymphocytes by the inventors, and it is human thioredoxin (hTRX). hTRX consists of 105 amino acids including cysteine residues at positions 32 and 35. In addition to TRX, a series of proteins possessing the active site: [-Cys-X-X-Cys-] (X: arbitrary amino acid) are present in cells, called the TRX superfamily, and considered to play important roles in intra- and extra-cellular redox control.

Intracellular TRX plays an important role in radical scavenging and the control of redox-related transcription factors, such as activator protein-1 (AP-1) and nuclear factor-kappa B (NF-κB) (AP-1 transcriptional activity is regulated by a direct association between thioredoxin and Ref-1 PNAS. 1997;94:3633-3638). hTRX controls the transmission of signals of p38 mitogen activating protein kinase (MAPK) and apoptosis signal regulating kinase-1 (ASK-1).

The inventors previously reported that TRX is released from cells and shows cytokine or chemokine activity (Circulating thioredoxin suppresses lipopolysaccharide-induced neutrophil chemotaxis PNAS. 2001; 98: 15143-15148) and extracellular TRX transfers into cells (Redox-sensing release of thioredoxin from T lymphocytes with negative feedback loops J. Immunol. 2004;172:442-448).

In the inflammatory process, leukocytes adhere to activated endothelial cells and are transferred from the blood circulation to injured tissue regions. The inventors have reported that TRX in blood circulation inhibited neutrophil infiltration in a mouse air pouch model (Nakamura H., et al. Proc. Natl. Acad. Sci. USA 98:15143-15148,2001.).

The above TRX superfamily includes macrophage migration inhibitory factor (MIF). MIF is an inflammatory cytokine that inhibits the random migration of macrophages to inflammatory regions, and plays an important role in systemic/local inflammation and immune responses (Nishihira J., J Interferon Cytokine Res, (2000) 20:751-762, Bucala R., FASEB J (1996) 7:19-24).

MIF has been reported to be produced by immunocompetent cells (lymphocytes and macrophages) and the pituitary in response to biological invasion (for example, stimulations by oxidative stress-inducing endotoxin, reactive oxygen, and ultraviolet light), located upstream of the inflammatory cytokine cascade, and control inflammatory reactions (by inducing expressions of other inflammatory cytokines) (Annual Reports in Medicinal Chemistry, vol. 33, P 24,1998, Advances in Immunology, vol. 66, P 197,1997). MIF is incorporated into cells by autocrine and paracrine mechanisms, and induces the production of inflammatory cytokines, TNF-alpha (α) and IL-1.

MIF has also been reported to play important roles in various biological reactions; (1) it promotes inflammation by inhibiting the anti inflammatory action of glucocorticoids:, (2) it is a T-lymphocyte activation-promoting factor:, (3) it inhibits p53 function:, and (4) it is involved in the proliferation and differentiation of adipocytes and cancer cells. (Bucala R., FASEB J (1996) 7:19-24, Bernhagen J., et al Nature 365: 756-759, 1993, Calandra T., et al Nature 377: 68-71,1995,).

However, because MIF exhibits an inflammatory action, its overproduction induces excess inflammatory reactions and causes various (inflammation-associated) disorders.

MIF induces diverse disorders (diseases). Such disorders typically include delayed allergy (type IV: cellular immunity reaction), such as rheumatoid arthritis. Related diseases have recently been increasingly reported, and its contribution to arteriosclerosis and endometriosis has been clarified. The marked elevation of MIF levels in alveolar lavage in adult respiratory distress syndrome (ARDS), urine in the rejection reaction of patients after renal transplantation, and serum in patients with acute myocardial infarction, diabetes, systemic lupus erythematosus, Crohn's disease, ulcerative colitis, and atopic dermatitis, compared to those in healthy subjects, has been reported.

Furthermore, the administration of anti MIF neutralizing antibodies improved the disease conditions in pathological animal models of nephritis, hepatitis, pneumonia, arthritis and endotoxin shock (International Journal of Molecular Medicine, vol. 2, P 17, 1998).

These findings indicate that the inflammatory action of MIF is involved in the aggravation of these pathologies.

In addition to these, there have been many reports on the relationship between MIF and various diseases (Makita H., et al. Am J Respir Crit Care Med 158:573-579,1998, Kobayashi S., et al. Hepatology 29:1752-1759, 1999, Mikulowaska A., et al. J Immunol 158:5514-5517, 1997, Ichiyama H., et al. Cytokine 26:187-194, 2004, Donnelly S. C., et al. Nat. Med. 3:320-323, 1997, Mizue Y, et al, Proc. Natl. Acad. Sci. USA 102:14410-14415,2005, Yang N., et al. Mol. Med. 4:413-424, 1998, Lan H. Y., et al. J Exp. Med. 185:1455-1465, 1997, Leung J. C., et al. Nephrol. Dial Transplant 19:36-45, 2003).

As described above, to control MIF is useful for reducing symptoms of various diseases. The inventors have discovered that TRX inhibits the activity of MIF and exhibits an anti inflammatory effect by directly binding to MIF, and proposed therapeutic and preventive agents using TRX as the active ingredient for disorders caused by MIF.

Regarding antibodies against TRX, a monoclonal antibody that specifically reacts with ADF, i.e., hTRX, has been prepared, and a measurement method of hTRX in human body fluids using this monoclonal antibody has been proposed (Japan Patent No. 3323258). Anti hTRX antibodies have also been reported to inhibit neutrophil migration to airway epithelial cells (Miller L. A., et al. J. Leukoc. Biol. 68:201-208, 2000).

There have been various reports regarding various effects of TRX or TRX superfamily. However, the effects of anti TRX antibodies, which do not belong to the TRX superfamily (e.g., their effects on disorders induced by MIF and vascular system disorders), have not been reported until now, although the anti TRX antibodies are closely related to TRX.

### SUMMARY OF INVENTION

### Problems to be Solved

The present invention provides a preventive or therapeutic agent with fewer side effects which is highly effective in preventing from or treating disorders induced by MIF and vascular system disorders.

### BRIEF DESCRIPTION OF THE DRAWINGS

FIG. 1 is the data showing flow cytometer analysis indicating TRX located on the surface of ATL2 cells.

FIG. 2 is the data showing that anti TRX antibody inhibits the cell internalization of MIF.

FIG. 3 is the data showing TNF-α concentration to indicate anti TRX antibody's inhibition of MIF activity.

FIG. 4 is the data showing the result of TRX and MIF binding analysis which was carried out with BIAcore2000 using surface plasmon resonance.

FIG. 5 is the data showing the result of flow cytometer analysis to indicate the expression of endogenous TRX on the surface of HUVECs.

FIG. 6 is the data showing anti Trx antibody inhibits the adherence of PMNs to HUVECs, which was analyzed by fluorescence microscope.

FIG. 7 is the data showing anti Trx antibody inhibits the adherence of PMNs to HUVECs, which was analyzed by quantification of fluorescence.

### DETAILED DESCRIPTION OF THE INVENTION

### Means to Solve the Problem

As a result of keen research, the present inventors found that anti TRX antibody polypeptide inhibited the cell internalization of MIF as well as adherence of leukocytes to vascular endothelial cells.

The present invention was completed after concluding that anti TRX antibody polypeptide was effective for disorders induced by MIF and vascular system disorders.

Therefore, one embodiment of the present invention is related to a preventive or therapeutic agent containing anti TRX antibody polypeptide as an active ingredient for disorders induced by macrophage migration inhibitory factor (MIF) and vascular system disorders.

Another embodiment of the present invention is related to the preventive or therapeutic agent wherein the anti TRX antibody inhibits migration (internalization) of the macrophage MIF into the cell.

Another embodiment of the present invention is related to the preventive or therapeutic agent wherein the anti TRX antibody inhibits adherence of leukocytes to vascular endothelial cells.

Another embodiment of the present invention is related to the preventive or therapeutic agent wherein the anti thioredoxin antibody is an anti human thioredoxin antibody against human thioredoxin.

Another embodiment of the present invention is related to the preventive or therapeutic agent wherein the anti human thioredoxin antibody is ADF11 antibody or ADF21 antibody.

Another embodiment of the present invention is related to the preventive or therapeutic agent wherein the disorders induced by macrophage MIF are caused by the inflammation in any one of cells or tissues selected from the group consisting of T cells, monocytes/macrophages, dendritic cells, mesangial cells, Sertoli's cells, keratinocytes, synovial cells, astrocytes, cancer cells, the mucosa and pituitary.

Another embodiment of the present invention is related to the preventive or therapeutic agent, wherein the disorders induced by macrophage MIF is any one selected from the group consisting of rheumatoid arthritis, arteriosclerosis, endometriosis, acute respiratory distress syndrome, bronchial asthma, renal transplantation-associated disorders, acute myocardial infarction, diabetes, systemic lupus erythematosus, Crohn's disease, ulcerative colitis, atopic dermatitis, nephritis, hepatitis, pneumonia, arthritis, IgA glomerulonephritis, endotoxin shock and infectious sepsis due to thrombocytopenia.

Another embodiment of the present invention is related to the preventive or therapeutic agent, wherein the vascular system disorders is any one selected from the group consisting of hyperlipidemia, atheromatous diseases, myocardial infarction and cerebral stroke.

The other embodiment of the present invention is related to a preventive or therapeutic preparation containing anti TRX antibody polypeptide as an active ingredient for disorders induced by macrophage migration inhibitory factor and vascular system disorders.

### Effect of the Invention

A preventive or therapeutic agent and preparation according to the present invention containing anti TRX antibody polypeptide as an active ingredient are effective in inhibiting inflammatory reaction induced by macrophage migration inhibitory factor (MIF) and tissue fibrosis caused by the inflammatory reaction, because anti TRX antibody inhibits cell internalization of macrophage MIF. Further present invention reduces inflammatory in a vascular induced by leukocytes, by inhibiting adherence of leukocytes to vascular endothelial cell.

Thus, the present invention may provide the preventive or therapeutic agent having preventive or therapeutic effect on disorders induced by macrophage MIF and vascular system disorders.

Further, preventive or therapeutic agent and preparation of the present invention comprise polypeptide of anti TRX antibody which is endogenous thiol protein expressed in vivo, as an active ingredient, and therefore they have fewer side effects and are highly safety.

### Preferred Embodiment

The inventors discovered that macrophage migration inhibitory factor (hereinafter MIF) is internalized into the activated T cells via TRX present on the cell surface, and this internalization can be inhibited by reaction between TRX and anti TRX antibody polypeptides. The inventors also discovered that leukocytes adhere to vascular endothelial cells via TRX present on the vascular endothelial cell membrane, and therefore such an adherence (i.e., adherence of leukocyte to vascular endothelial cell) can be inhibited by the reaction of TRX with anti TRX antibody polypeptides.

Anti TRX antibodies exhibit inhibitory effects on MIF-induced inflammatory reactions, tissue fibrosis associated with these inflammatory reactions, and inflammation induced by adherence of leukocyte to vascular endothelium cells, based on which we concluded that anti TRX antibodies are useful for the treatment of MIF-induced and vascular system disorders, and completed this invention.

Embodiments of preventive or therapeutic agent for disorders caused by MIF and vascular system disorders using anti TRX antibody polypeptides as an active ingredient are closely explained below.

### [Anti TRX Antibodies]

Anti TRX antibodies of this invention are antibodies against TRX.

TRX includes human thioredoxin (hTRX) and other molecules belonging to the TRX family, such as polypeptides possessing -Cys-Gly-Pro-Cys-, -Cys-Pro-Tyr-Cys-, -Cys-Pro-His-Cys-, and -Cys-Pro-Pro-Cys- in the active center from residues 32 to 35.

Among these TRX molecules, ‘hTRX’ and ‘TRX2’ (mitochondria-specific thioredoxin) possessing -Cys-Gly-Pro-Cys- in the active center are preferred, and hTRX is more preferable.

The origin of TRX is not limited, as far as the molecules have the above active centers, but it may be TRX derived from animals including humans (ADF derived from animals including humans), bacterial TRX such as E. coli-derived TRX, yeast TRX, polypeptides with human ADF activity (human ADFP), and human and E. coli glutaredoxin. Human and yeast TRX are preferred.

The term of “polypeptide” used here may include polypeptide having any molecular weight, for example, proteins.

Accordingly, for anti TRX antibodies, those against hTRX and TRX2 possessing -Cys-Gly-Pro-Cys- in the active center, i.e., anti hTRX and anti TRX2 antibodies, are desired, and more preferably, anti hTRX antibodies.

The anti TRX antibodies according to the present invention may be polyclonal antibodies, but monoclonal antibodies that specifically react with TRX are preferred in the present invention.

MIF is internalized into the activated T cells via TRX present on the cell surface, and anti TRX antibody polypeptides of this invention are considered to inhibit MIF internalization into cells by reacting with TRX on the cell surface. Leukocytes adhere to vascular endothelial cells via TRX on surface of vascular endothelial cells membrane, and anti TRX antibody polypeptides of this invention inhibit the adherence by reacting with TRX on vascular endothelial cells. Accordingly, anti TRX antibody polypeptides may be the whole antibody molecule including the Fab and Fc regions and partial antibody molecules possessing the Fab region recognizing TRX epitopes, as far as these molecules specifically react with TRX.

Anti hTRX antibodies may include monoclonal antibodies that specifically react with ADF, which is hTRX. For example, monoclonal antibodies ADF11 produced by the hybridoma cell line ADF11 (accession number FERM P-13025), and ADF21 produced by the hybridoma cell line ADF21 (accession number FERM P-13026) (refer to Japan patent No. 3323258).

ADF11 and 12 belong to the same subclass, IgG1 (κ), but recognize different epitopes.

The above anti TRX antibody polypeptides may be used alone or in combination of 2 or more polypeptides.

Regarding the route of administration of the preventive or therapeutic agents of this invention, the agents may be administered both orally and non-orally, and the route may be appropriately selected by clinicians. The active ingredients, which are anti TRX antibody polypeptides, may be used alone or administered with generally applied carriers.

Oral dosage forms of the preventive or therapeutic agents of this invention include solid preparations (e.g., tablets, pills, powders, coated tablets, granular powder and capsules), liquid forms (e.g., liquid preparations, suspensions, emulsions and syrup), inhalants (e.g., aerosol, atomizer and nebulizer) and liposome-mounted preparations.

The preventive or therapeutic agent or preparation of this invention is practically applied as pharmaceutical forms using pharmacologically competent carriers with the above active ingredients. Examples of the pharmaceutical carriers include binders, disintegrators, surfactants, absorption-promoting agents, humectants, adsorbents, lubricants, fillers, volume-increasing agents, moisturizers, antiseptics, stabilizers, emulsifiers, solubilizers, salts for adjusting osmotic pressure, diluents (e.g., buffers) and excipient. These are appropriately selected according to the dosage form of the preparations.

For tablet shaping, the carriers described above may be used, for example, excipients (e.g., lactose, sucrose, sodium chloride, glucose, urea, starch, calcium carbonate, kaolin, crystalline cellulose, silicic acid and potassium phosphate), binders (e.g., water, ethanol, propanol, simple syrup, glucose solution, starch solution, gelatin solution, carboxymethyl cellulose, hydroxypropyl cellulose, methylcellulose and polyvinylpyrrolidone), disintegrators (e.g., sodium carboxymethylcellulose, calcium carboxymethylcellulose, low-substituted hydroxypropylcellulose, dry starch, sodium alginate, agar powder, laminaran powder, sodium bicarbonate and calcium carbonate), surfactants (e.g., polyoxyethylene sorbitan esters of fatty acids, sodium lauryl sulfate and monoglyceride stearate), disintegration inhibitors (e.g., sucrose, stearin, cacao butter and hydrogenated oil), absorption-promoting agents (e.g., quaternary ammonium salt and sodium lauryl sulfate), humectants (e.g., glycerin and starch), adsorbents (e.g., starch, lactose, kaolin, bentonite and colloid silicic acid), and lubricants (e.g., purified talc, stearates, boric acid powder and polyethylene glycol).

Tablets may be coated, as needed, such as sugar-coated, gelatin-capsulated, enteric-coated and film-coated, as needed. The tablets may be double- or multi-layered ones, when coated with two or more layers.

Regarding pill shaping, the carriers described above may be used, for example, excipient such as glucose, lactose, starch, cacao butter, hydrogenated plant oil, kaolin and talc, binders such as acacia powder, powdered tragacanth, gelatin and ethanol, and disintegrators such as laminaran and agar.

Non-oral dosage forms of the preventive or therapeutic agents of this invention may be injection form (e.g., liquid preparations, emulsions, and suspensions) used for intravenous, subcutaneous, intracutaneous, intramuscular and intraperitoneal injections, liquid preparations (e.g., eye and nasal drops), suspensions, emulsions, drip infusions and inhalants (e.g., aerosols and powder inhalants).

For the preparation of liquids, emulsions, and suspensions of the preventive or therapeutic agents of this invention, it is preferable that these are disinfected and isotonic with blood. For this, water, ethyl alcohol, macrogol, propylene glycol, ethoxyisostearyl alcohol, polyoxyisostearyl alcohol and polyoxyethylene sorbitan esters of fatty acids, e.t.c, may be used as the diluents.

For such cases, an adequate amount of sodium chloride, glucose, or glycerin for isotonic solution preparation may be added to the agent or preparation of this invention.

Conventional solubilizers, buffers and soothing agents may also be used.

When the preventive or therapeutic agents of this invention are liquid preparations, they may be stored after the removal of water by freezing and freeze-drying. Freeze-dried preparations are dissolved again with distilled water for injection at the time of use.

When the preventive or therapeutic agents of this invention are used as inhalants, known general additives for inhalants may be used for their preparation. Examples of the additives include propellants, solid excipients (e.g., sucrose, lactose, glucose, mannite and sorbit), liquid excipients (e.g., inactive liquid including propylene glycol), binders (e.g., methylcellulose, hydroxypropyl cellulose, polyvinylpyrrolidone, polyethylene glycol and sucrose), lubricants (e.g., magnesium stearate, light anhydrous silicic acid, talc and sodium lauryl sulfate), preservatives (e.g., sodium benzoate, sodium bisulfite, methylparaben and propylparaben), stabilizers (e.g., citric acid and sodium citrate), suspending agents (e.g., methylcellulose, polyvinylpyrrolidone, lecithin and triolein sorbitan), dispersing agents (e.g., surfactants), solvents (e.g., water), isotonic agents (e.g., sodium chloride), pH-adjusting agents (e.g., sulfuric acid and hydrochloric acid), and solubilizers (e.g., ethanol).

The preventive or therapeutic agent of this invention may contain coloring agents, preservatives, perfumes, flavoring agents, sweeteners and other agents.

The effective dose of anti TRX antibody polypeptides may be easily determined by those skilled in referring to the existing techniques, for example, about 0.1-5 mg/kg per day per adult, preferably, about 0.5-1 mg/kg.

This dose may be administered by single dosing or divided into several times per day. It is preferable to adjust the dosing frequency depending on the dosage form, patient's gender and age and the degree of diseases.

The agent or preparations containing anti TRX antibody polypeptides as the active ingredients of this invention may also be used as beverages and food products, for which plant materials may be added to the extracts described above.

Examples of beverages and food products include health foods, nutrient-supplementing foods (e.g., balanced nutrient diets and supplements), functional nutrient foods, specified foods for health use and foods for those with diseases. The manufacturing methods of these food products are not specified, and they may be manufactured by methods used by skilled persons.

Examples of beverages and food products include sweets such as gum, candies, gummies, tablet sweets, cookies, cakes, chocolates, ice cream, jelly, mousse, pudding, biscuits, cornflakes, chewable tablets, wafers and senbei (rice crackers seasoned with soy sauce), beverages such as carbonated drinks, refreshing drinks, lactic drinks, coffee, tea, fruit juice, nutrient-enriched beverages, alcohol and mineral water, powdered beverages such as powdered juice and soup, balanced nutrient diets, supplements such as the ones in powder, capsules and tablet forms, seasonings such as dressings and sauces, bread, noodles, products made with boiled fish paste such as kamaboko, and furikake (dried food for sprinkling on rice).

### [Macrophage Migration Inhibitory Factor (MIF)]

MIF is present in various animal species including humans. It is a 12.3-kDa protein consisting of 114 amino acid residues, and possesses the redox active domain: Cys-X-X-Cys- motif. Therefore it belongs to the TRX superfamily.

MIF is expressed in not only lymphocytes but also various organs such as the brain and kidney. It is strongly expressed in uriniferous tubular epithelial cells in the kidney, and also expressed in actively proliferating basal membrane cells in the skin and cornea.

MIF-expressing cells and tissues may include T cells, monocytes/macrophages, dendritic cells, mesangial cells, uriniferous tubular epithelial cells, corneal epithelial cells, hepatocytes, ova, Sertoli's cells, keratinocytes, osteoblasts, synovial cells, adipocytes, astrocytes, cancer cells, the mucosa and pituitary.

MIF is incorporated by activated T cells via TRX on the cell surface. Anti TRX antibody polypeptides of this invention inhibit the cell internalization (incorporation) of MIF by reacting with TRX, and therefore MIF-induced inflammation and inflammation-associated fibrosis are inhibited.

Leukocytes adhere to vascular endothelial cells via TRX on the cell membrane surface. Anti TRX antibody polypeptides of this invention inhibit adherence of leukocyte to vascular endothelial cells by reacting with TRX, and therefore inflammation induced by adherence of leukocyte to vascular endothelial cells is inhibited.

Since the active ingredients of the preventive or therapeutic agent of this invention are anti TRX antibody polypeptides, it inhibits MIF-induced inflammation and adherence of leukocyte to vascular endothelial cells, which is useful for the prevention and treatment of MIF-induced diseases and vascular system disorders.

Diseases (disorders) for which the preventive or therapeutic agent of this invention is applicable, i.e., disorders induced by MIF, are explained here. MIF-induced disorders are not specified as far as they are MIF-induced ‘inflammation’ and ‘tissue fibrosis’ resulting from inflammatory reactions. The disorders may include those described below.

### [Disorders]

Disorders for which the preventive or therapeutic agent of this invention is applicable are those induced by MIF and vascular system disorders. The term, ‘disorder’, is herein used in its wide definition to include ‘diseases’.

MIF-induced disorders may include inflammation associated with MIF-expressing cells and tissues, such as T cells, monocytes/macrophages, dendritic cells, mesangial cells, uriniferous tubular epithelial cells, corneal epithelial cells, hepatocytes, ova, Sertoli's cells, keratinocytes, osteoblasts, synovial cells, adipocytes, astrocytes, cancer cells, the mucosa and pituitary.

These disorders may include those directly induced by an inflammatory action of MIF in the cells and tissues expressing MIF described above and those induced in various cells and tissues induced by indirect actions of MIF.

Typical disorders induced by MIF may include delayed allergy, rheumatoid arthritis, arteriosclerosis, endometriosis, acute respiratory distress syndrome (ARDS), bronchial asthma, renal transplantation-associated disorders, acute myocardial infarction, diabetes, systemic lupus erythematosus, Crohn's disease, ulcerative colitis, atopic dermatitis, nephritis (particularly, rapidly progressive glomerulonephritis), IgA glomerulonephritis, hepatitis (particularly, acute hepatitis), pulmonary disorders (e.g., disorders due to infiltration of neutrophil and monocyte into the alveoli and induction of them to bronchial alveolar lavage, and disorders caused by pulmonary hemorrhage), pneumonia (e.g., BCG-LPS-induced acute pneumonia), arthritis, acticular rheumatism (particularly, type II collagen arthritis and adjuvant arthritis), endotoxin shock and infectious sepsis due to thrombocytopenia.

Vascular system disorders may include hyperlipidemia, atheromatous diseases, myocardial infarction and cerebral stroke. Hyperlipidemia-associated diabetic nephropathy may be also included in the hyperlipidemia.

The invention will be specifically explained referring to the examples below, but it is not limited to these examples.

### EXAMPLE 1

[Confirmatory Experiment of Existence of TRX on the Activated T Cell Surface]

It was confirmed whether TRX exists on the activated T cell surface or not.

### [Materials]

ATL2 cells; human adult T-cell leukemia cell strain Jurkat T cells; human acute T-cell leukemia cell strain (provided by Dr. YODOI Junji, Institute for Virus Research)

a. Anti TRX antibody (ADF 11 antibody) (Redox Bioscience Inc.)

b. ALEXA FLUOR 488 protein labeling kit (Invitrogen Corp.)

c. FACS Flow Solution (Becton Dickinson)

d. Flow cytometer; FACS Calibur (Becton Dickinson)

e. Flowjo Software (Tree Star, Inc.)

### [Method]

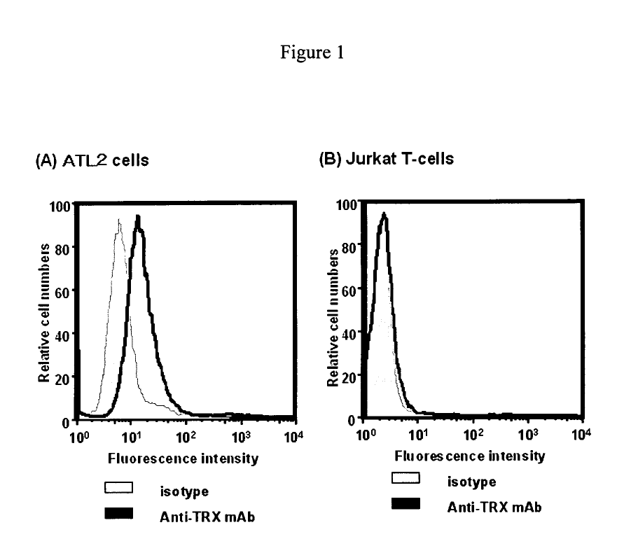

Fluorescence-labeled anti TRX antibody 100 ng/ml was prepared with ALEXA FLUOR 488 labeling kit. The resulting antibody was mixed with ATL2 cells (1×10⁶ cell/well), and then left at 4° C. for 30 minutes. Then, cells were collected, washed with FACS FLOW Solution, measured with FACS Calibur, and then analyzed with Flowjo Software. Control experiment was carried out in the same way as the above except for using Jurkat T cells as a non-activated T cell. The result is shown in FIG. 1.

FIG. 1(A) shows TRX, upon which anti TRX antibody influences (i.e., to which anti TRX antibody binds), is localized on the cell surfaces of ATL2 (Activated T cells). On the other hand, it is confirmed from the FIG. 1(B) that anti TRX antibody does not act on (i.e., does not bind to) the Jurkat T cells (i.e., non-activated T cells), and therefore TRX is not localized. (see FIG. 1(B).)

### EXAMPLE 2

[Confirmatory Experiment of Anti TRX Antibody Inhibiting the Cell Internalization of MIF]

Effects of anti TRX antibody on the cell internalization of MIF were examined.

### [Materials]

ATL2 cells; human adult T-cell leukemia cell strain Recombinant MIF (MIF); His-tagged recombinant MIF was expressed in Escherichia coli cells by using expression vector pQE30 (QIAGEN), and column-purified with MagneHis™ Protein Purification System (Promega Corp.).

a. Anti TRX antibody (ADF 11 antibody) (Redox Bioscience Inc.)

b. Anti His-tagged antibody (Promega Corp.).

c. Mouse IgG (BD Pharmingen)

### [Method]

ATL2 cells were cultured with RPMI 1640 medium in the 75 cm³ flask, wherein the RPMI 1640 medium contained 10% FCS, 100 U/ml penicillin, and 100 μg/ml streptomycin.

ATL2 cells were added to 24 well plates (1×10⁶ cell/well). The anti TRX antibody having final concentration of 0, 1 and 10 μg/ml and the recombinant MIF (25 μg/ml) were also added thereto. Then, the plate was cultured with RPMI 1640 medium under the condition of 0.5% CO₂ at 37° C. for 24 hours.

After cells were collected, the precipitation was mixed with SDS-PAGE sample buffer, and then electrophoresed on a 15% SDS-PAGE gel. After the electrocataphoresis, cell internalization of MIF was examined by performing a Western blotting with anti His-tagged antibody.

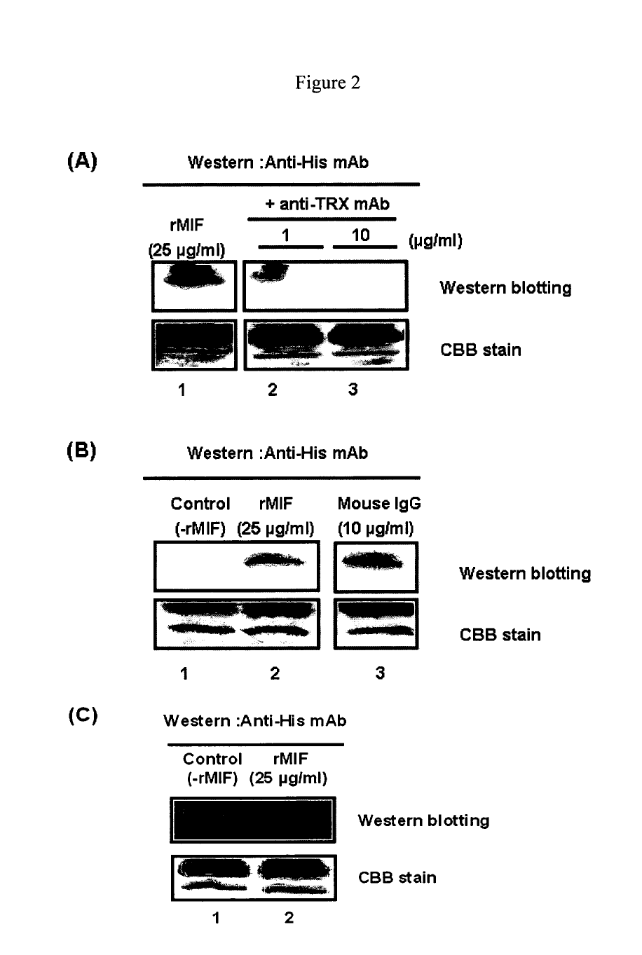

Control experiment was carried out in the same way as the above except for using mouse IgG instead of anti TRX antibody. The result is shown in FIG. 2.

As indicated in FIG. 2(A), internalization of MIF into ATL2 cells is inhibited in the presence of anti TRX antibody. FIG. 2 (B) shows internalization of MIF into ATL2 cells was not inhibited in the presence of mouse IgG which was used as control.

From the results of EXAMPLEs 1 and 2, it is assumed that MIF was internalized by cells via TRX which exists on the cell surface of the activated T cells. Therefore anti TRX antibody is assumed to inhibit cell internalization of MIF by acting on the TRX.

In addition, it was not confirmed an internalization of the recombinant MIF in the Jurkat T cells (FIG. 2(C)).

As described above, cell internalization of the recombinant MIF was shown in the cells which have TRX on the surface thereof. Therefore it is suggested that anti TRX antibody may inhibit inflammatory response, because anti TRX antibody has influence on TRX which exists on the cell surface and, subsequently, inhibits the cell internalization of MIF.

### EXAMPLE 3

### [Confirmatory Experiment of Anti TRX Antibody's Inhibition of MIF Activity]

Anti TRX antibody's inhibition of MIF activity was confirmed by measuring a concentration of inflammatory cytokine TNF-α.

### [Materials]

RAW 264.7 cells: mouse-derived macrophage cell strains (provided by Dr. ISHII Yasuyuki, RIKEN, Research Center for Allergy and Immunology)

a. Recombinant MIF (MIF) (ATGEN CO., LTD.)

b. Lipopolysaccharide (LPS) (SIGMA)

c. TNF-α ELISA kit; R & D (SYSTEMS)

### [Method]

RAW 264.7 cells were cultured with RPMI 1640 medium in the 75 cm³ flask, wherein the RPMI 1640 medium contained 10% FCS, 100 U/ml penicillin, and 100 μg/ml streptomycin.

RAW 264.7 cells were added to 24 well plates (1×10⁶ cell/well). MIF having final concentration of 0 and 5 μg/ml and anti TRX antibody (0, 500 and 1000 ng/ml) were also added thereto. Then, the plate was left under the condition of 5% CO₂ at 37° C. for 4 hours.

Further, LPS (0, 100 ng/ml) was added to the plate, and the plate was left under the condition of 5% CO₂ at 37° C. for 4 hours. Then, medium was collected.

Amount of the generated TNF-alpha (α) was measured by Duo Set ELISA Development system mouse TNF-α kit.

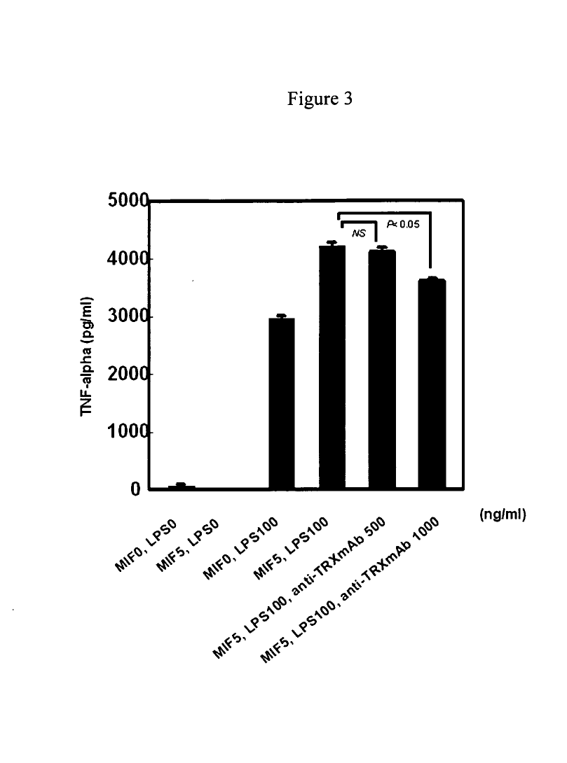

The method for the measurement was performed according to the protocol attached to the kit. The result of the measurement is shown in FIG. 3.

FIG. 3 shows that anti TRX antibody having the concentration of 1000 ng/ml significantly inhibits the TNF-α generation derived from MIF, while anti TRX antibody having the concentration 500 ng/ml does not significantly inhibit the same. Thus, it was confirmed that anti TRX antibody concentration-dependently inhibited such a generation.

From the results of EXAMPLEs 1 to 3, it is found that anti TRX antibody inhibits cell internalization of MIF by acting on TRX which exists on the surface of the activated T cell.

Therefore, anti TRX antibody may inhibit or reduce inflammatory response because anti TRX antibody, consequently, inhibits cell internalization of MIF, and then generation of TNF-alpha is increased.

As a result, anti TRX antibody is effective for disorder caused by MIF.

### EXAMPLE 4

### [Analysis of the Intermolecular Interaction by Using Surface Plasmon Resonance Technique on TRX and MIF]

Recombinant TRX and MIF were measured with BIA core 2000 according to the surface plasmon resonance technique to analyze affinity of TRX and MIF.

### [Materials]

Recombinant TRX (Ajinomoto Co., Inc.)
Recombinant MIF (prepared in the EXAMPLE 2)
Buffer; 10 mM HEPES, pH 7.4, 150 mM NaCl, 5 mM MgCl₂, 1 mM EDTA, 1 mM DDT, 0.005% tween20

a. BIA core 2000 (GE Healthcare)

b. Sensor Chip NTA (GE Healthcare)

### [Method]

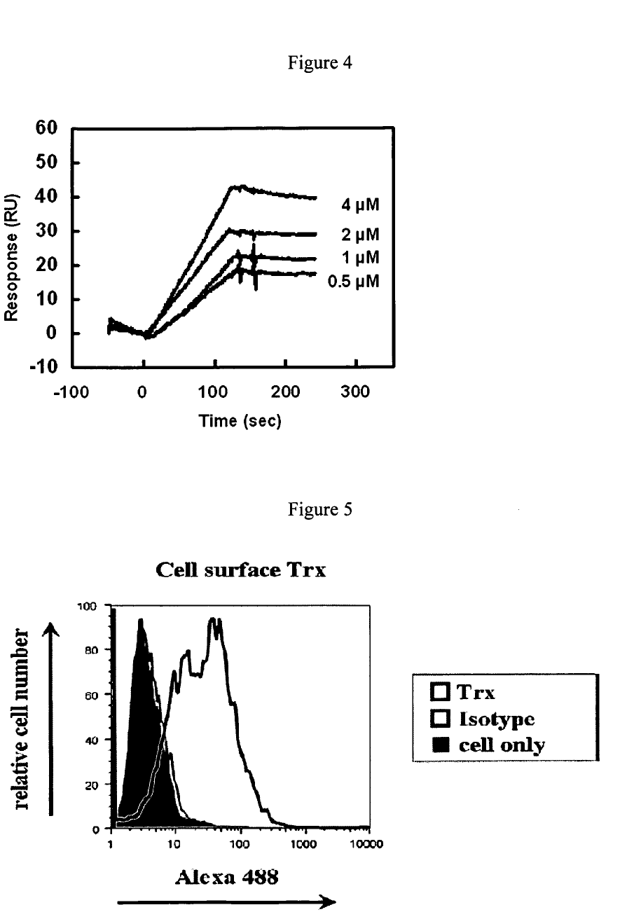

BIA core 2000 was used for the analysis. Recombinant MIF was bound to Sensor Chip NTA by 1100 RU at the flow rate (10 μL/min) and measurement temperature (25° C.). With the sensor chip bound to the recombinant MIF and the recombinant TRX (0.5˜4 μM), binding analysis was carried out at the flow rate (20 μL/min) and measurement temperature (25° C.). For control, binding analysis was carried out with the recombinant TRX (0.5˜4 μM) and sensor chip to which the recombinant MIF was not bound, and the other procedures were the same as the above. Affinity analysis of TRX and MIF was performed with BIA evaluation software 3.1. The result is shown in FIG. 4.

As shown in FIG. 4, TRX was bound to MIF in a concentration-dependent manner. kₐ=1.2×10² M⁻¹S⁻¹, k_{d}=1.19×10⁻⁶ M⁻¹ S⁻¹, K_{D}=9.96×10⁻⁹M were obtained as the result of the analysis from 1:1 binding with drifting baseline model analysis-method.

The above-result shows TRX may act as receptor of MIF like, because there isn't so much of a difference between the above-result and the value (K_{D}=9×10⁻⁹ M) resulting from previously-reported binding analysis for MIF and CD74 which is known as a receptor of MIF.

### EXAMPLE 5

### [The Experiment of Anti TRX Antibody's Inhibition of the Adherence of Leukocytes to Endothelial Cells]

Anti TRX antibody's effect on the adherence of leukocytes to vascular endothelial cells was examined.

### [Materials]

Anti TRX antibody (ADF 11 antibody) (Redox Bioscience Inc.,) Human umbilical vein endothelial cells (HUVECs; Cambrex Corp.) EGM-2 medium (Cambrex Corp.)

Monopoly resolving medium (Dainippon-Sumitomo pharmaceutical Inc.,)
a. Alexa conjugated anti TRX mAb; prepared using an Alexa Fluor 488 protein labeling kit (Invitrogen Corp.)
b. Cell wash buffer (Becton Dickinson.)
c. Phosphate-buffered saline; PBS
d. Flow cytometer (FACS Calibur) (Becton Dickinson)
e. Software (Cell Quest) (Becton Dickinson).
f. 2′,7′-bis-(2-carboxyethyl)-5-(and-6)-carboxyfluorescein acetoxymethyl ester (Invitrogen Corp.)
g. 6-well plate (Asahi Techno Glass Corp.,)
h. BZ-8000 fluorescence microscope (KEYENCE Corp.,)
i. Calcein AM (Invitrogen Corp.)
j. Fluorescence microplate reader (Gemini EM)

### [Method]

### [Cell Preparation]

HUVECs were maintained with EGM-2 medium in a humidified atmosphere of 5% CO₂/95% air at 37° C.

Human peripheral blood polymorphonuclear cells (PMNs) were isolated from freshly drawn heparinized blood, which was obtained from healthy volunteers, using Monopoly resolving medium.

### [Confirmatory Experiment of Expression of Endogenous TRX on the Surface of Vascular Endothelial Cells]

HUVECs were mixed with a saturating concentration of the indicated Alexa-conjugated anti TRX mAb, then suspended in cell wash buffer and incubated for 30 min on ice, in the dark. They were then washed with an excess of phosphate-buffered saline (PBS) and analyzed using a flow cytometer. Data acquisition was done with the software Cell Quest. The result of the Flow cytometer analysis is shown in FIG. 5.

As shown in FIG. 5, Expression of TRX was observed on the normal HUVEC surface.

### [Experiment of Adherence of Leukocytes to Vascular Endothelial Cells]

This experiment was carried out by a known method (Nakamura H. et al. Proc. Natl. Acad. Sci. USA 98:15143-15148, 2001.) with some modification.

HUVECs and PMNs were treated with anti TRX antibody (20 g/ml) for 30 min before the start of the adherence experiment.

The PMNs were labeled with 2′, 7′-bis-(2-carboxyethyl)-5-(and-6)-carboxyfluorescein acetoxymethyl ester and were incubated with serum free RPMI 1640 medium for 1 h at 37° C.

The labeled PMNs (5×10⁶ cells/ml) were washed with an excess of RPMI 1640 medium containing 10% serum, and then incubated with 1 g/ml LPS for 3 h, after which they were washed with an excess of HBSS containing 2% serum.

The PMNs were co-cultured with normal HUVEC monolayers in a glass bottomed 6-well plate.

After the co-culturing, non-adherent PMNs were discarded by washing them carefully with an excess of RPMI 1640 medium containing 10% serum. The plates were photographed with a BZ-8000 fluorescence microscope.

The control which was not treated by anti TRX antibody was experimented as the same way as the above.

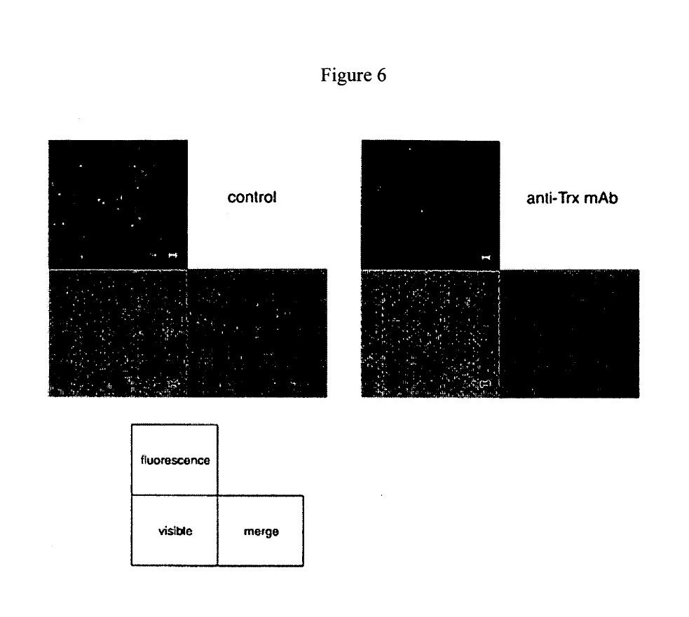

The result is shown in FIG. 6. (Scale is 50 μm.)

It was observed from FIG. 5 and FIG. 6 that PMNs (leukocyte) adhered to the endothelial cells by TRX expressed on the surface of HUVECs (vascular endothelial cells). It is thought that Anti TRX antibody inhibits the adherence of LPS-stimulated PMNs to normal HUVECs by reacting with TRX.

### [Cell Binding Analysis]

This experiment was carried out by a known method (Nakamura H., et al. Proc. Natl. Acad. Sci. USA 98:15143-15148, 2001 and Roy S., et al. Methods Enzymol 300:395-401, 1999) with some modification.

HUVECs and PMNs were treated with anti TRX antibody for 30 min before the start of the experiment.

PMNs (5×10⁶ cells/ml) were labeled with 5 μM calcein AM, according to the manufacturer's instructions.

The labeled PMNs were pre-treated with 1 μg/ml LPS for 3 h, then washed with an excess of HBSS containing 2% serum.

Next they were co-cultured with normal HUVEC monolayers in microtiter plates.

After the co-culturing, non-adherent PMNs were discarded by washing them carefully with an excess of RPMI 1640 medium containing 10% serum.

The bound cells were analyzed by a quantification of fluorescence (excitation at 485 nm and emission at 538 nm) with a fluorescence microplate reader.

The control which was not treated by anti TRX antibody was experimented as the same way as the above.

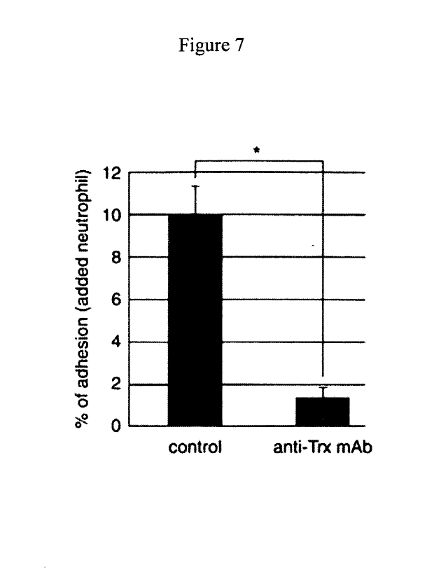

The result is shown in FIG. 7. The differences between control and anti TRX-mAb were evaluated with the student t-test for comparison. (p values<0.05, n=3)

As shown in FIG. 7, it is observed that anti TRX antibody significantly inhibits PMNs activated by LPS from adhering to normal HUVECs.

As shown in the result of the above-experiment for confirming expression of endogenous TRX on the surface of vascular endothelial cells, adherence of leukocytes to endothelial cells, and cell binding analysis, it was recognized that anti TRX antibody inhibited the adherence of leukocytes to vascular endothelial cells by having an influence on TRX existing on the surface of the vascular endothelial cells.

Therefore, anti TRX antibody is considered to be effective for the vascular system disorders.

1-9. (canceled)

10. A preventive or therapeutic agent containing anti thioredoxin antibody polypeptide as an active ingredient for disorders induced by macrophage migration inhibitory factor and vascular system disorders.

11. The preventive or therapeutic agent according to claim 10, wherein the anti thioredoxin antibody polypeptide inhibits migration of macrophage migration inhibitory factor into the cell.

12. The preventive or therapeutic agent according to claim 10, wherein the anti thioredoxin antibody polypeptide inhibits adherence of leukocyte to vascular endothelial cell.

13. The preventive or therapeutic agent according to claim 11, wherein the anti thioredoxin antibody is an anti human thioredoxin antibody against human thioredoxin.

14. The preventive or therapeutic agent according to claim 12, wherein the anti thioredoxin antibody is an anti human thioredoxin antibody against human thioredoxin.

15. The preventive or therapeutic agent according to claim 13, wherein the anti human thioredoxin antibody polypeptide is ADF11 antibody or ADF21 antibody.

16. The preventive or therapeutic agent according to claim 14, wherein the anti human thioredoxin antibody polypeptide is ADF11 antibody or ADF21 antibody.

17. The preventive or therapeutic agent according to claim 11, wherein the disorders induced by the macrophage migration inhibitory factor are caused by the inflammation in any one of cells or tissues selected from the group consisting of T cells, monocytes/macrophages, dendritic cells, mesangial cells, Sertoli's cells, keratinocytes, synovial cells, astrocytes, cancer cells, the mucosa and pituitary.

18. The preventive or therapeutic agent according to claim 11, wherein the disorders induced by macrophage migration inhibitory factor is any one selected from the group consisting of rheumatoid arthritis, arteriosclerosis, endometriosis, acute respiratory distress syndrome, bronchial asthma, disorders associated with renal transplantation, acute myocardial infarction, diabetes, systemic lupus erythematosus, Crohn's disease, ulcerative colitis, atopic dermatitis, nephritis, hepatitis, pneumonia, arthritis, IgA glomerulonephritis, endotoxin shock and infectious sepsis due to thrombocytopenia.

19. The preventive or therapeutic agent according to claim 12, wherein the vascular system disorders is any one selected from the group consisting of hyperlipidemia, atheromatous diseases, myocardial infarction and cerebral stroke.

20. A preventive or therapeutic preparation containing anti thioredoxin antibody polypeptide as an active ingredient for disorders induced by macrophage migration inhibitory factor and vascular system disorders.
